(19) United States
(12) Patent Application Publication
(10) Pub. No.: US 2025/0381501 A1
(43) Pub. Date: Dec. 18, 2025

(54) ROSE HYDROSOL AND PREPARATION METHOD THEREOF

(71) Applicant: Shanxi University, Taiyuan City (CN)

(72) Inventor: Xiuqing YANG, Taiyuan City (CN)

(21) Appl. No.: 18/980,464

(22) Filed: Dec. 13, 2024

(30) Foreign Application Priority Data
Jun. 14, 2024 (CN) 202410773094.X

(51) Int. Cl. B01D 11/02; A61K 8/9789; A61K 36/738; A61Q 19/00

(52) U.S. Cl. CPC B01D 11/0211; A61K 8/9789; A61K 36/738; A61Q 19/00; B01D 11/0223; B01D 11/0288; A61K 2236/31; A61K 2236/331; A61K 2236/37; A61K 2800/10; A61K 2800/81

(57) ABSTRACT

Disclosed are a rose hydrosol and a preparation method thereof. The preparation method includes subjecting a rose flower to microwave-assisted extraction, collecting a resulting steam, and subjecting the resulting steam to condensation to obtain the rose hydrosol. A microwave method is used for the first time to prepare the rose hydrosol, which could directly extract water and rose aroma components from fresh rose petals with a fast extraction speed but free of long-time distillation.

### CROSS REFERENCE TO RELATED APPLICATION

This patent application claims the benefit and priority of Chinese Patent Application No. 202410773094X filed with the China National Intellectual Property Administration on Jun. 14, 2024, the disclosure of which is incorporated by reference herein in its entirety as part of the present application.

### TECHNICAL FIELD

The present disclosure belongs to the technical field of natural product extraction, and specifically relates to a rose hydrosol and a preparation method thereof.

### BACKGROUND

Rose hydrosol is an extract from rose flowers, with natural ingredients and a light fragrance. Rose hydrosol is soluble in water and has functions such as replenishing moisture and moisturizing. It could be applied to human skin without irritation or harm, and could maintain the moisture in the skin for a long time. Rose hydrosol could also have anti-wrinkle and acne-removing effects. Spraying rose hydrosol on the skin could not only fully cleanse the skin, but also effectively kill bacteria, which makes it easier for the skin to absorb nutrients in the rose hydrosol. Rose hydrosol is a water solution that is closer to water. Compared with rose essential oil, it is more skin-friendly and easier to be absorbed by human skin. Rose hydrosol is not only widely used in cosmetics, but also in tobacco flavors, foods and medicines.

Rose hydrosol is usually obtained by distilling rose flowers and water. For example, Chinese patent publication No. CN103655372A discloses a method for preparing a rose hydrosol by distillation, but the rose hydrosol prepared by the method smells sour and rotten. Currently, rose hydrosol is mostly prepared by distillation, or obtained through the water remained during the distillation and extraction process for essential oils. Both methods require a long time of distillation. Both methods require a long time of distillation, which not only makes the rose hydrosol have the taste of sweet potatoes, but also the remaining rose fragrance in the remaining flower cannot be utilized. If it is not handled in time, it would cause environmental pollution. Therefore, seeking a new method for extracting rose hydrosol has become inevitable.

### SUMMARY

In view of this, an object of the present disclosure is to provide a rose hydrosol and a preparation method thereof. In the present disclosure, microwave extraction is used to prepare rose hydrosol, which does not require long-term distillation. The extracted rose hydrosol exhibits a strong aroma and almost the same taste as rose flowers. Moreover, the residue is dried rose flowers containing essential oils, which are not easily rotten, and rose essential oils could be further extracted therefrom.

To achieve the above object, the present disclosure provides the following technical solutions:

The present disclosure provides a method for preparing a rose hydrosol, including the following steps:

subjecting a rose flower to microwave-assisted extraction, collecting a resulting steam, and subjecting the resulting steam to condensation to obtain the rose hydrosol.

In some embodiments, the rose flower has a moisture content of 83% to 92%.

In some embodiments, the rose flower is in a thickness of a single layer during the microwave-assisted extraction.

In some embodiments, the microwave-assisted extraction is conducted at a power of 6 kW to 10 kW for 2 min to 35 min.

In some embodiments, the rose flower during the microwave-assisted extraction has a maximum temperature of 65° C. to 85° C.

In some embodiments, the condensation is performed by using a condensing water at a temperature of 10° C. to 15° C.

In some embodiments, the method is conducted by using a device including:
a cracking chamber configured for the microwave-assisted extraction;
a condensation system connected to the cracking chamber; and
a draft fan arranged at a connection between the cracking chamber and the condensation system and configured to introduce a vaporized material into the condensation system, the draft fan having a work frequency of 18 Hz to 30 Hz.

In some embodiments, the cracking chamber is equipped with a conveyor belt to convey the rose flower, and the conveyor belt has a speed of 0 m/min to 2.5 m/min.

The present disclosure further provides a rose hydrosol prepared by the method as described in the above technical solutions.

Compared with the prior art, some embodiments of the present disclosure have the following beneficial effects:

The present disclosure provides a method for preparing a rose hydrosol, including the following steps: subjecting a rose flower to microwave-assisted extraction, collecting a resulting steam, subjecting the resulting steam to condensation to obtain the rose hydrosol. In the present disclosure, a microwave method is used for the first time to prepare the rose hydrosol, which could directly extract water and rose aroma components from fresh rose petals, with a fast extraction speed but free of long-time distillation. The extracted rose hydrosol has a strong aroma and almost the same taste as rose flowers. Moreover, the method of the present disclosure does not need to add any ingredients (including water), and realizes green extraction and pollution-free discharge. The by-product (dried rose flowers) could be directly used to extract rose essential oil, or directly used as food stuffing in the food field, which realizes a comprehensive and complete utilization of rose flowers.

The method for preparing the rose hydrosol according to the present disclosure is time-saving, uses less cooling water, and consumes less energy in the extraction process. In addition to the rose hydrosol, the remaining dried rose flower could be further utilized to extract rose essential oil, which has very good social and economic significance for the overall development of roses.

### BRIEF DESCRIPTION OF THE DRAWINGS

In order to more clearly illustrate the embodiments of the present disclosure or the technical solutions in the prior art, the drawings used in the examples will be briefly introduced below. Obviously, the drawings described below are only some embodiments of the present disclosure. For a person of ordinary skill in the art, other drawings could be obtained based on these drawings without creative efforts.

The FIGURE shows a schematic diagram of a microwave extraction equipment used in Examples, where numeral references are listed below: 1 refers to a conveyor belt, 2 refers to a bracket, 3 refers to a cracking chamber (with three cracking chambers connected in series), 4 refers to a cracking chamber door, 5 refers to a material inlet, and 6 refers to a draft fan.

### DETAILED DESCRIPTION OF THE EMBODIMENTS

The present disclosure provides a method for preparing a rose hydrosol, including the following steps:

subjecting a rose flower to microwave-assisted extraction, collecting a resulting steam, and subjecting the resulting steam to condensation to obtain the rose hydrosol.

In the present disclosure, unless otherwise specified, all the materials and devices used are commercially available products in the art.

In some embodiments of the present disclosure, the rose flower is freshly picked, which contains a rosebud. In some embodiments, the rose flower has full and uncompressed petals.

In some embodiments of the present disclosure, a color of the rose flower is yellow or white. In some embodiments, the rose flower is rose petals with a receptacle and calyx removed beforehand. In some embodiments, the rose flower has a moisture content of 83% to 92%, preferably 88% to 91%.

In some embodiments of the present disclosure, the microwave-assisted extraction is conducted at a power of 6 kW to 10 KW, and preferably 8 kW to 10 kW. In some embodiments, the microwave-assisted extraction is conducted for 2 min to 35 min, preferably 15 min to 30 min, and even more preferably 20 min. The microwave-assisted extraction provides in addition to thermal effects, also polarization and vibration, which breaks down cells and provides vaporization conditions with thermal effects.

In some embodiments of the present disclosure, the rose flower during the microwave-assisted extraction has a maximum temperature of 65° C. to 85° C., preferably 70° C. to 80° C. In some embodiments, the temperature of the rose flower is raised at a rate of 15° C./min to 20° C./min during the first 3 minutes of extraction.

In some embodiments of the present disclosure, the method is conducted by using a device including: a cracking chamber configured for the microwave-assisted extraction; a condensation system which is in connection with the cracking chamber; and a draft fan arranged at a connection between the cracking chamber and the condensation system and configured to introduce a vaporized material into the condensation system. In some embodiments, there are three cracking chambers, each having a length of 1 m. In some embodiments, the cracking chamber is equipped with a conveyor belt (having a width of 0.8 m) to convey the rose flower. In some embodiments, the conveyor belt has a speed of 0 m/min to 0.12 m/min, preferably 0.06 m/min to 0.1 m/min, and more preferably 0.08 m/min. In some embodiments, a weight of the rose flower on the convey belt is in a range of 4.5 kg to 12 kg, preferably 6.4 kg to 8 kg.

In some embodiments, the condensation is performed by using a condensing water at a temperature of 10° C. to 15° C., and preferably 12° C. to 15° C. The condensing water fully condenses a vaporized extract to obtain a condensed liquid. In some embodiments, the draft fan has a work frequency of 18 Hz to 30 Hz, and preferably 22 Hz to 25 Hz. The steam generated by microwave-assisted extraction of the rose flower is introduced by the draft fan into the condensation system to undergo condensation and then be collected to the condenser. When the temperature of the rose flower is 65° C., the frequency of the draft fan is adjusted to 18 Hz to 30 Hz. When the temperature of the rose flower is 75° C., the frequency of the draft fan is adjusted to 20 Hz to 25 Hz.

In some embodiments of the present disclosure, a time from microwave-assisted extraction to the outflow of the rose hydrosol from the condenser is in a range of 2 min to 6 min, preferably 2 min to 3 min. In some embodiments, after the rose hydrosol flows out of the condenser, the microwave-assisted extraction is continued for 12 min to 20 min, preferably 13 min to 15 min.

The method for preparing the rose hydrosol according to the present disclosure has the advantages of green extraction process, short time consumption, no addition of any additives (including water), small amount of cooling water consumption and low energy consumption.

The present disclosure further provides a rose hydrosol prepared by the method as described in the above technical solutions.

The rose hydrosol according to the present disclosure has bright and clear color, and it has a sweet smell of the rose flower, and strong and long-lasting aroma, and is easy to store. The rose hydrosol according to the present disclosure could be used directly for drinking, skin care or compounding.

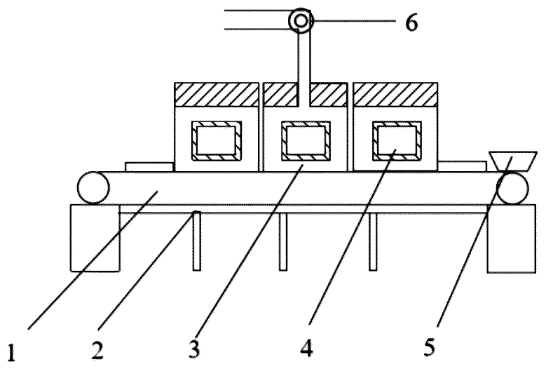

In some examples according to the present disclosure, the microwave extraction equipment includes/composed of: cracking chambers for microwave processing (3 cracking chambers connected in series, each having a length of 1 m, equipped with a conveyor belt for conveying materials); a condensation system connected to the cracking chambers; and a draft fan installed above the cracking chamber, wherein the draft fan is configured to introduce gas into the condensation system for condensation and a resulting system is collected to the condenser, as shown in FIG. 1. In FIG. 1, 1 refers to the conveyor belt, 2 refers to a bracket, 3 refers to the cracking chamber (with three cracking chambers connected in series), 4 refers to a cracking chamber door, 5 refers to a material inlet, and 6 refers to the draft fan.

In the present disclosure, the above microwave extraction device is used to achieve continuous production of rose hydrosol.

To further illustrate present disclosure, the rose hydrosol and the preparation method thereof according to the present disclosure will be described in detail below in conjunction with FIGURES and examples, which could not be considered as limiting the scope of the present disclosure, however.

### Comparative Example 1

2.5 kg of freshly picked rose flowers, water, and common salt, according to a formula of 10 parts by weight of rose flowers, 60 parts by weight of water and 0.5 parts by weight of common salt, were added into a distillation kettle, and a resulting mixture were then subjected to distillation at a distillation temperature of 60° C. for 10 hours to obtain a distillate. The distillate was cooled to a temperature of 15° C. to obtain a condensate. The condensate was then collected to obtain the rose hydrosol.

### Example 1

In this example, a rose hydrosol was prepared by a method as follows:

4.5 kg of raw materials were used for preparing the rose hydrosol. Rose flowers with yellow receptacles and calyces were removed through screening. The condensation system was turned on. When the outlet temperature of condensing water reached 10° C. to 12° C., fresh rose flowers were conveyed to the cracking chamber of the microwave extraction equipment with a crawler conveyor. After the fresh rose flowers entered the cracking chamber, the speed of the conveyor belt was adjusted to 0, and meanwhile a microwave magnetron was turned on, and a power of the microwave magnetron was adjusted to 6 kW.

When the temperature of the fresh rose flowers reached 65° C. to 70° C., the draft fan was turned on, and the frequency of the draft fan was adjusted to 22 Hz. After 2 min, a rose hydrosol was collected from the condensation system, and after 20 min, the collection was finished, to obtain the rose hydrosol.

### Example 2

In this example, a rose hydrosol was prepared by a method as follows:

4.5 kg of raw materials were used for preparing the rose hydrosol. Rose flowers with yellow receptacles and calyces were removed through screening. Fresh rose flowers were conveyed to the cracking chamber of the microwave extraction equipment with a crawler conveyor. The condensation system was turned on. When the outlet temperature of condensing water reached 12° C. to 15° C., the conveyor belt was started to a speed of 0.1 m/min, and meanwhile a microwave magnetron was turned on, and a power of the microwave magnetron was adjusted to 8 kW.

When the temperature of the fresh rose flowers reached 70° C. to 75° C., the draft fan was turned on, and the frequency of the draft fan was adjusted to 22 Hz. After 2 min, a rose hydrosol was collected from the condensation system, and after 25 min, the collection was finished, to obtain a rose hydrosol.

### Example 3

In this example, a rose hydrosol was prepared by a method as follows:

6.4 kg of raw materials were used for preparing the rose hydrosol. Rose flowers with yellow receptacles and calyces were removed through screening. Fresh rose flowers were conveyed to the cracking chamber of the microwave extraction equipment with a crawler conveyor. The condensation system was turned on. When the outlet temperature of condensing water reached 12° C. to 15° C., the conveyor belt was started to a speed of 0.08 m/min, and meanwhile a microwave magnetron was turned on, and a power of the microwave magnetron was adjusted to 8 kW.

When the temperature of the fresh rose flowers reached 75° C. to 80° C., the draft fan was turned on, and the frequency of the draft fan was adjusted to 22 Hz. After 2 min, a rose hydrosol was collected from the condensation system, and after 30 min, the collection was finished, to obtain the rose hydrosol.

### Example 4

In this example, a rose hydrosol was prepared by a method as follows:

8 kg of raw materials were used for preparing the rose hydrosol. Rose flowers with yellow receptacles and calyces were removed through screening. Fresh rose flowers were conveyed to the cracking chamber of the microwave extraction equipment with a crawler conveyor. The condensation system was turned on. When the outlet temperature of condensing water reached 12° C. to 15° C., the conveyor belt was started to a speed of 0.1 m/min, and meanwhile a microwave magnetron was turned on, and a power of the microwave magnetron was adjusted to 8 kW.

When the temperature of the fresh rose flowers reached 75° C. to 80° C., the draft fan was turned on, and the frequency of the draft fan was adjusted to 22 Hz. After 3 min, the rose hydrosol was collected from the condensation system, and after 30 min, the collection was finished, to obtain the rose hydrosol.

### Example 5

In this example, a rose hydrosol was prepared by a method as follows:

12 kg of raw materials were used for preparing the rose hydrosol. Rose flowers with yellow receptacles and calyces were removed through screening. Fresh rose flowers were conveyed to the cracking chamber of the microwave extraction equipment with a crawler conveyor. The condensation system was turned on. When the outlet temperature of condensing water reached 10° C. to 12° C., the conveyor belt was started to a speed of 0.1 m/min, and meanwhile a microwave magnetron was turned on, and a power of the microwave magnetron was adjusted to 10 kW.

When the temperature of the fresh rose flowers reached 80° C. to 85° C., the draft fan was turned on, and the frequency of the draft fan was adjusted to 25 Hz. After 3 min, the rose hydrosol was collected from the condensation system, and after 30 min, the collection was finished, to obtain the rose hydrosol.

Extractions of the rose hydrosol in Comparative Example 1 and Examples 1-5 are shown in Table 1:

TABLE 1
Extractions of the rose hydrosol in Comparative
Example 1 and Examples 1-5

| | Mass | | Extraction % | |
|---|---|---|---|---|
| | of raw | Extraction | (Extraction | |
| | materials | amount of | amount/Fresh | |
| | of rose | rose | raw materials | |
| | flowers | hydrosol | of the rose | Sensory |
| Group | (kg) | (kg) | flowers) | Evaluation |
| Comparative | 2.5 | 0.875 | 35 | Bright and clear |
| Example 1 | | | | color; a sweet |
| | | | | smell of rose |
| | | | | flowers, and a |
| | | | | sweet potato |
| | | | | flavor and a light |
| | | | | sour taste |
| Example 1 | 4.5 | 2.475 | 55 | Bright and clear |
| | | | | color; a sweet |
| | | | | smell of rose |
| | | | | flowers, and |
| | | | | strong and |
| | | | | long-lasting |
| | | | | aroma |
| Example 2 | 4.5 | 2.7 | 60 | Bright and clear |
| | | | | color; a sweet |
| | | | | smell of rose |
| | | | | flowers, and |
| | | | | strong and |
| | | | | long-lasting |
| | | | | aroma |
| Example 3 | 6.4 | 3.9 | 65 | Bright and clear |
| | | | | color; a sweet |
| | | | | smell of rose |
| | | | | flowers, and |
| | | | | strong and |
| | | | | long-lasting |
| | | | | aroma |
| Example 4 | 8 | 4.24 | 53 | Bright and clear |
| | | | | color; a sweet |
| | | | | smell of rose |
| | | | | flowers, and |
| | | | | strong and |
| | | | | long-lasting |
| | | | | aroma |
| Example 5 | 12 | 6 | 50 | Bright and clear |
| | | | | color; a sweet |
| | | | | smell of rose |
| | | | | flowers, and |
| | | | | strong and |
| | | | | long-lasting |
| | | | | aroma |

The rose hydrosol as obtained by the examples according to the present disclosure has bright and clear color, and it has a sweet smell of rose flowers, strong and long-lasting aroma, and is easy to store. The rose hydrosol obtained in the comparative example has a sweet potato flavor and a light sour taste and is difficult to store. Moreover, the extraction rate of the rose hydrosol in the present disclosure is significantly higher than that in the comparative example.

Although the examples described above have provided a detailed description of the present disclosure, they are only a part of, rather than all of the embodiments of the present disclosure. Other embodiments that could be obtained according to the embodiments of the present disclosure without creative efforts shall fall within the scope of the disclosure.

What is claimed is:

1. A method for preparing a rose hydrosol, comprising:
subjecting a rose flower to microwave-assisted extraction;
collecting a resulting steam; and
subjecting the resulting steam to condensation to obtain the rose hydrosol.

2. The method according to claim 1, wherein the rose flower has a moisture content of 83% to 92%.

3. The method according to claim 1, wherein the rose flower is in a thickness of a single layer during the microwave-assisted extraction.

4. The method according to claim 2, wherein the rose flower is in a thickness of a single layer during the microwave-assisted extraction.

5. The method according to claim 1, wherein the microwave-assisted extraction is conducted at a power of 6 kW to 10 kW for 2 min to 35 min.

6. The method according to claim 2, wherein the microwave-assisted extraction is conducted at a power of 6 kW to 10 kW for 2 min to 35 min.

7. The method according to claim 1, wherein the rose flower during the microwave-assisted extraction has a maximum temperature of 65° C. to 85° C.

8. The method according to claim 1, wherein the condensation is performed by using a condensing water at a temperature of 10° C. to 15° C.

9. The method according to claim 1, wherein the method is conducted by using a device comprising:
a cracking chamber configured for the microwave-assisted extraction;
a condensation system connected to the cracking chamber; and
a draft fan arranged at a connection between the cracking chamber and the condensation system and configured to introduce a vaporized material into the condensation system, the draft fan having a work frequency of 18 Hz to 30 Hz.

10. The method according to claim 8, wherein the method is conducted by using a device comprising:
a cracking chamber configured for the microwave-assisted extraction;
a condensation system connected to the cracking chamber; and
a draft fan arranged at a connection between the cracking chamber and the condensation system and configured to introduce a vaporized material into the condensation system, the draft fan having a work frequency of 18 Hz to 30 Hz.

11. The method according to claim 9, wherein the cracking chamber is equipped with a conveyor belt to convey the rose flower, and the conveyor belt has a speed of 0 m/min to 2.5 m/min.

12. A rose hydrosol prepared by the method according to claim 1.

13. The rose hydrosol according to claim 12, wherein the rose flower has a moisture content of 83% to 92%.

14. The rose hydrosol according to claim 12, wherein the rose flower is in a thickness of a single layer during the microwave-assisted extraction.

15. The rose hydrosol according to claim 12, wherein the microwave-assisted extraction is conducted at a power of 6 kW to 10 kW for 2 min to 35 min.

16. The rose hydrosol according to claim 12, wherein the rose flower during the microwave-assisted extraction has a maximum temperature of 65° C. to 85° C.

17. The rose hydrosol according to claim 12, wherein the condensation is performed by using a condensing water at a temperature of 10° C. to 15° C.
